(19) United States
(12) Patent Application Publication
(10) Pub. No.: US 2024/0229014 A1
(43) Pub. Date: Jul. 11, 2024

(54) SYSTEM FOR THE DIRECTED EVOLUTION OF BIOMOLECULES IN MULTI-BODY COMPLEXES

(71) Applicant: Massachusetts Institute of Technology, Cambridge, MA (US)

(72) Inventors: Emma J. Chory, Boston, MA (US); Kevin Esvelt, Newton, MA (US); Vainavi Mukkamala, West Windsor, NJ (US)

(73) Assignee: Massachusetts Institute of Technology, Cambridge, MA (US)

(21) Appl. No.: 18/408,191

(22) Filed: Jan. 9, 2024

Related U.S. Application Data
(60) Provisional application No. 63/479,092, filed on Jan. 9, 2023.

(51) Int. Cl. C12N 15/10

(52) U.S. Cl. CPC C12N 15/1058; C12N 15/1055; C12N 15/1086

(57) ABSTRACT

The invention, in part, includes systems and methods for conducting and optimizing continuous evolution of molecules capable of forming multi-body complexes, such as but not limited to evolution of molecules capable of forming three- and four-molecule multi-body complexes.

### RELATED APPLICATIONS

This application claims benefit under 35 U.S.C. § 119(e) of U.S. Provisional application Ser. No. 63/479,092 filed Jan. 9, 2023, the content of which is incorporated herein by reference in its entirety.

### GOVERNMENT INTEREST

This invention was made with government support under grant number 1F32CA247274-01 awarded by the National Institutes of Health. The Government has certain rights in the invention.

### FIELD OF THE INVENTION

This disclosure relates generally to systems, components, and methods for evolving biomolecules that are involved in multi-body complexes.

### BACKGROUND OF THE INVENTION

Continuous evolution of biomolecules provides one of the most rapid and effective means of evolving protein functions (Esvelt et al. 2011; Miller et al. 2020). However, to date, the most effective means of protein evolution with continuous evolution have been limited to proteins with enzymatic function, such as polymerases (Esvelt et al. 2011), proteases (Dickinson et al. 2014), and synthetases (Bryson et al. 2017). This is in part, because compared to other forms of directed evolution, continuous evolution introduces an additional factor that cannot readily be optimized in high-throughout, long term, continuous, exponential growth of bacteria. Prior methods of continuous evolution of proteins involved coupling the biological activity of a protein of interest to the expression of a protein required for replication of an organism, such as a bacteriophage, or replication of a yeast plasmid. When evolving proteins that are involved in a specific binding interaction (such as an antibody-antigen interaction, small-molecule dimerizer, or protein-protein interaction), the coupling of activity to gene expression is generally achieved by fusing the evolving proteins of interest to two components that need to be in close proximity in order to produce their function.

Prior methods have been limited due, in part, to an inability to develop new evolution “circuits” that require the careful titration of, or large amounts of chemically synthesized small molecules, such as chemical inducers. Recently, a method for the high-throughput maintenance of bacteria growing at log phase was developed that allows growth of bacteria for many days thereby permitting one to achieve equilibrium prior to dose-optimization (Chory et al. 2021). Further, a system was developed that enables the continuous evolution in low-volume cultures (DeBenedictis et al. 2021). Irrespective of such advances, effective and efficient systems and methods for continuous evolution of multi-body complexes such protein-protein (ternary complexes) and protein-small molecule protein binding (quaternary complexes) have not been attained.

### SUMMARY OF ASPECTS OF THE INVENTION

According to an aspect of the invention, a method of evolving molecules of multi-body complexes is provided, the method including: preparing an evolution circuit including a plurality of molecules capable of forming a multi-body complex; forming the multi-body complex including the plurality of molecules in the prepared evolution circuit; determining for each of the plurality of molecules, one or more equilibrium conditions in the prepared evolution circuit that produce an equimolar ratio of each of the plurality of molecules in the evolution circuit; selecting one or more evolution conditions for the plurality of molecules based at least in part on the one or more equilibrium conditions determined for each of the plurality of molecules; maintaining the selected one or more evolution conditions; and evolving one or more of the molecules of the multibody complex under the maintained one or more selected evolution conditions. In some embodiments, the selected one or more evolution conditions are the same as the one or more determined equilibrium condition. In some embodiments, the selected one or more evolution conditions are different than the one or more determined equilibrium conditions. In some embodiments, the plurality of molecules capable of forming the multi-body complex includes three molecules. In some embodiments, the plurality of molecules capable of forming the multi-body complex comprise one or more of proteins and small molecules. In some embodiments, the evolution circuit is prepared with a liquid-handling robot. In some embodiments, a means for preparing the evolution circuit includes an automated, high-throughput turbidostat method on a liquid-handling robot. In some embodiments, the method also includes culturing a cell capable for use for protein evolution in the automated, high-throughput turbidostat on the liquid-handling robot. In some embodiments, the cultured cell is bacteria+phage or yeast. In some embodiments, the method also includes maintaining a selected density of the cultured cell, optionally wherein the selected density is maintained using a feedback controller in the liquid-handling robot. In some embodiments, the method also includes equilibrating growth of the cultured cell; contacting a plurality of portions of the equilibrated cell culture with one or more combinations of concentrations of a first-molecule inducing agent and a second molecule-inducing agent; inducing production of the first molecule of the multi-body complex and the second molecule of the multi-body complex; and identifying one or more combinations of concentrations of the first-molecule-inducing agent and the second molecule-inducing agent that result in kinetic equilibrium of the first molecule and second molecules in the evolution circuit. In some embodiments, the method also includes contacting a portion of the equilibrated cell culture with one of the identified combinations of concentrations of the first-molecule-inducing agent and the second molecule-inducing agent that results in kinetic equilibrium of the first molecule and second molecules in the evolution circuit with a predetermined concentration of the third molecule of the multi-body complex that results in kinetic equilibrium of the third molecule; and initiating evolution of one or more of the first, second, and third molecules in the evolution circuit. In some embodiments, the cultured cell is a bacterium and the method further includes contacting the cultured cell including the first, second, and third molecules of the multi-body complex maintained at kinetic equilibrium with a phage. In some embodiments, initiating evolution includes initiating mutagenesis in the cultured cells in the evolution circuit including the first, second, and third molecule at their respective kinetic equilibriums. In some embodiments, each of the first, second, and third molecules of the multi-body complex is not maintained at its kinetic equilibrium when the evolution is initiated. In some embodiments, two of the molecules capable of forming the multi-body complex are inducible proteins. In some embodiments, the two inducible proteins are expressed in plasmids in the cultured cell. In some embodiments, one of the proteins is an exogenously expressed first protein fused to a DNA binding domain. In some embodiments, one of the proteins is an exogenously expressed second protein fused to a DNA-directed RNA polymerase (RNAP) subunit. In some embodiments, the one of the molecules capable of forming the multi-body complex is a DNA-directed RNA polymerase (RNAP) expressed by the cultured cell. In some embodiments, the RNAP expressed by the cultured cell is endogenous to the cultured cell. In some embodiments, the method also includes determining an amount of the first, second, and third molecules in the evolution circuit, wherein a means of determining the amount includes including at least one independently selected detectable label on one or more of the first, second, and third molecules. In some embodiments, the identifying of the one or more conditions resulting in kinetic equilibrium of the first, second, and third molecules includes: contacting the cultured cell with a plurality of amounts of the first and second molecule-inducing agents, respectively; determining an amount of the first molecule inducing agent that induces the kinetic equilibrium of the first molecule; and determining an amount of the second molecule inducing agent that induces the kinetic equilibrium of the second molecules. In some embodiments, one or more of the first and second molecule inducing-agents is a small-molecule inducing agent. In some embodiments, the small molecule inducing agent is a promoter. In some embodiments, the promoter is an environment-specific promoter, optionally is a heat shock promoter, a phage shock promoter, a quorum sensing promoter. In some embodiments, one or more of the first and second molecule inducing agents is light. In some embodiments, the one or more conditions are one or more of: an amount of the inducing agent, identity of the inducing agent, timing of an initial contact of the cultured cell with the inducing agent, length of time of contact of the cultured cell with the inducing agent. In some embodiments, the produced kinetic equilibrium of the plurality of molecules results in an equimolar ratio of the plurality of molecules capable of forming the multi-body complex. In some embodiments, the multibody complex further includes one or more detectable reporter. In some embodiments, the detectable reporter includes one or more of a luminescence reporter, a fluorescence reporter, and a phage protein reporter. In some embodiments, a means for the determining of one or more conditions in the prepared evolution circuit capable of producing kinetic equilibrium of each of the plurality of molecules, includes detecting the detectable reporter. In some embodiments, one or more of the molecules forming the multi-body complex is a protein and preparing the evolution circuit includes expressing the one or more proteins in the cultured cell. In some embodiments, the method also includes contacting the cultured cell in the evolution circuit with one or more independently selected inducible promoter to induce expression of the one of more of the proteins of the multi-body complex. In some embodiments, an induced protein is RNAP. In some embodiments, RNAP is endogenously expressed by the cultured cell. In some embodiments, the plurality of molecules is four molecules. In some embodiments, the plurality of molecules is four molecules and a first molecule and second molecule of the molecules capable of forming the multi-body complex are inducible proteins. In some embodiments, the inducible proteins are expressed in plasmids in the cultured cell. In some embodiments, the first molecule is an exogenously expressed first protein fused to a DNA binding domain. In some embodiments, the second molecule is an exogenously expressed second protein fused to a DNA-directed RNA polymerase (RNAP) subunit. In some embodiments, a third molecule of the molecules capable of forming the multi-body complex is a molecule capable of dimerizing the first and second molecules capable of forming the multi-body complex. In some embodiments, a fourth molecule of the molecules capable of forming the multi-body complex is a DNA-directed RNA polymerase (RNAP) expressed by the cultured cell. In some embodiments, the RNAP expressed by the cultured cell is endogenous to the cultured cell.

According to another aspect of the invention, a method of evolving one or more molecules of a multi-body complex is provide, the method comprising: (a) preparing an evolution circuit comprising a plurality of molecules capable of forming a multi-body complex; (b) determining for each of the plurality of molecules, one or more equilibrium conditions in the prepared evolution circuit capable of producing an equimolar ratio of each of the plurality of molecules in the evolution circuit; (c) selecting one or more evolution conditions for the plurality of molecules based at least in part on the one or more equilibrium conditions determined for each of the plurality of molecules; (d) maintaining the selected one or more evolution conditions; and (e) evolving one or more of the molecules of the multibody complex under the maintained one or more selected evolution conditions. In some embodiments, the method also includes forming the multi-body complex including the plurality of molecules in the prepared evolution circuit. In some embodiments, the method also includes forming the multi-body complex comprising one or more of the evolved molecules. In some embodiments, the selected one or more evolution conditions are the same as the one or more determined equilibrium condition. In some embodiments, the selected one or more evolution conditions are different than the one or more determined equilibrium conditions. In some embodiments, the plurality of molecules capable of forming the multi-body complex includes three molecules. In some embodiments, the plurality of molecules capable of forming the multi-body complex comprise one or more of proteins and small molecules. In some embodiments, the evolution circuit is prepared with a liquid-handling robot. In some embodiments, a means for preparing the evolution circuit includes an automated, high-throughput turbidostat method on a liquid-handling robot. In some embodiments, the method also includes culturing a cell capable for use for protein evolution in the automated, high-throughput turbidostat on the liquid-handling robot. In some embodiments, the cultured cell is bacteria+phage or yeast. In some embodiments, the method also includes maintaining a selected density of the cultured cell, optionally wherein the selected density is maintained using a feedback controller in the liquid-handling robot. In some embodiments, the method also includes equilibrating growth of the cultured cell; contacting a plurality of portions of the equilibrated cell culture with one or more combinations of concentrations of a first-molecule inducing agent and a second molecule-inducing agent; inducing production of the first molecule of the multi-body complex and the second molecule of the multi-body complex; and identifying one or more combinations of concentrations of the first-molecule-inducing agent and the second molecule-inducing agent that result in kinetic equilibrium of the first molecule and second molecules in the evolution circuit. In some embodiments, the method also includes contacting a portion of the equilibrated cell culture with one of the identified combinations of concentrations of the first-molecule-inducing agent and the second molecule-inducing agent that results in kinetic equilibrium of the first molecule and second molecules in the evolution circuit with a predetermined concentration of the third molecule of the multi-body complex that results in kinetic equilibrium of the third molecule; and initiating evolution of one or more of the first, second, and third molecules in the evolution circuit. In some embodiments, the cultured cell is a bacterium and the method further includes contacting the cultured cell including the first, second, and third molecules of the multi-body complex maintained at kinetic equilibrium with a phage. In some embodiments, initiating evolution includes initiating mutagenesis in the cultured cells in the evolution circuit including the first, second, and third molecule at their respective kinetic equilibriums. In some embodiments, each of the first, second, and third molecules of the multi-body complex is not maintained at its kinetic equilibrium when the evolution is initiated. In some embodiments, two of the molecules capable of forming the multi-body complex are inducible proteins. In some embodiments, the two inducible proteins are expressed in plasmids in the cultured cell. In some embodiments, one of the proteins is an exogenously expressed first protein fused to a DNA binding domain. In some embodiments, one of the proteins is an exogenously expressed second protein fused to a DNA-directed RNA polymerase (RNAP) subunit. In some embodiments, the one of the molecules capable of forming the multi-body complex is a DNA-directed RNA polymerase (RNAP) expressed by the cultured cell. In some embodiments, the RNAP expressed by the cultured cell is endogenous to the cultured cell. In some embodiments, the method also includes determining an amount of the first, second, and third molecules in the evolution circuit, wherein a means of determining the amount includes including at least one independently selected detectable label on one or more of the first, second, and third molecules. In some embodiments, the identifying of the one or more conditions resulting in kinetic equilibrium of the first, second, and third molecules includes: contacting the cultured cell with a plurality of amounts of the first and second molecule-inducing agents, respectively; determining an amount of the first molecule inducing agent that induces the kinetic equilibrium of the first molecule; and determining an amount of the second molecule inducing agent that induces the kinetic equilibrium of the second molecules. In some embodiments, one or more of the first and second molecule inducing-agents is a small-molecule inducing agent. In some embodiments, the small molecule inducing agent is a promoter. In some embodiments, the promoter is an environment-specific promoter, optionally is a heat shock promoter, a phage shock promoter, a quorum sensing promoter. In some embodiments, one or more of the first and second molecule inducing agents is light. In some embodiments, the one or more conditions are one or more of: an amount of the inducing agent, identity of the inducing agent, timing of an initial contact of the cultured cell with the inducing agent, length of time of contact of the cultured cell with the inducing agent. In some embodiments, the produced kinetic equilibrium of the plurality of molecules results in an equimolar ratio of the plurality of molecules capable of forming the multi-body complex. In some embodiments, the multibody complex further includes one or more detectable reporter. In some embodiments, the detectable reporter includes one or more of a luminescence reporter, a fluorescence reporter, and a phage protein reporter. In some embodiments, a means for the determining of one or more conditions in the prepared evolution circuit capable of producing kinetic equilibrium of each of the plurality of molecules, includes detecting the detectable reporter. In some embodiments, one or more of the molecules forming the multi-body complex is a protein and preparing the evolution circuit includes expressing the one or more proteins in the cultured cell. In some embodiments, the method also includes contacting the cultured cell in the evolution circuit with one or more independently selected inducible promoter to induce expression of the one of more of the proteins of the multi-body complex. In some embodiments, an induced protein is RNAP. In some embodiments, RNAP is endogenously expressed by the cultured cell. In some embodiments, the plurality of molecules is four molecules. In some embodiments, the plurality of molecules is four molecules and a first molecule and second molecule of the molecules capable of forming the multi-body complex are inducible proteins. In some embodiments, the inducible proteins are expressed in plasmids in the cultured cell. In some embodiments, the first molecule is an exogenously expressed first protein fused to a DNA binding domain. In some embodiments, the second molecule is an exogenously expressed second protein fused to a DNA-directed RNA polymerase (RNAP) subunit. In some embodiments, a third molecule of the molecules capable of forming the multi-body complex is a molecule capable of dimerizing the first and second molecules capable of forming the multi-body complex. In some embodiments, a fourth molecule of the molecules capable of forming the multi-body complex is a DNA-directed RNA polymerase (RNAP) expressed by the cultured cell. In some embodiments, the RNAP expressed by the cultured cell is endogenous to the cultured cell.

### BRIEF DESCRIPTION OF THE DRAWINGS

FIG. 1 is a schematic diagram showing certain embodiments of multi-component systems used in continuous evolution for protein-protein (ternary complex) or protein-small molecule-protein binding (quaternary complex).

FIG. 2A-B provides schematic diagrams illustrating impact of a “hook-effect” on multi-body complexes. FIG. 2A illustrates results when two components are maintained at a fixed concentration. FIG. 2B illustrates results when one component is maintained at a fixed concentration.

FIG. 3 provides a graph illustrating that any concentration that is greater than the local maximum will not favor evolution.

FIG. 4 is a schematic diagram illustrating effect of identifying optimal conditions for evolving components (molecules) that form a multi-body complex. In this example, there are three components (molecules) that can form the multi-body complex (A, B, and C). The diagram provides a summary of the differential equations that govern the binding kinetics of a continuous evolution reaction. The “sweet spot” is defined as the concentrations of components A, B, and C that give the maximum effect. These concentrations are variable on time-dependent parameters, including the growth rate of bacteria, the infection rate of phage, the induction rate of any given gene (equations 2-4). Methods of the invention are used to allow the system to reach equilibrium, thus eliminating the time-dependent variables that can fluctuate over time, prior to equilibrating. In doing so, an accurate concentration can be determined that allow for the optimum parameters for an evolution circuit when in continuous flow.

FIG. 5 provides a flow diagram of an embodiment of a high-throughput robotic method used to optimize expression of the “hook-effect” components being used in a multi-body evolution.

FIG. 6 provides graphs showing the amount of reporter being expressed in an 8×12 grid while being induced at growth equilibrium. Vertical dashed lines indicate induction time points, dark grey dotted lines indicate doses where hook effect is detrimental to evolution.

FIG. 7 provides graphs of the amount of reporter being expressed in an 8×12 grid following both growth and expression equilibrium before induction and following induction by 1 or 2 different inducers with two different inducible promoters, in which it is identified that the optimum dose is orders of magnitude lower than the standard working concentration for inducible promoters.

FIG. 8 provides a grid plot visualization of phage enrichment in a 6×8 matrix, representing the results of an experiment conducted with two different small molecules across varying concentrations. Each cell in the grid corresponds to a unique combination of concentrations for the two molecules. The data shown are averages of duplicate measurements. Phage enrichment is quantified in terms of plaque-forming units per milliliter (PFU/mL) and is color-coded for ease of interpretation. The color gradient represents the degree of phage enrichment, with warmer colors indicating higher levels of enrichment. This visualization highlights the optimal concentration ranges for maximal phage propagation under the given experimental conditions.

FIG. 9 provides a bar plot illustrating the fraction of the population for three different mixtures of antibodies, each having distinct binding affinities (0.5 nM, 0.6 nM, and 60 nM), within a 4×4 concentration matrix. The experiment involved sequencing these mixtures using long-read nanopore sequencing to accurately deconvolute the proportion of each antibody type in the population under varying conditions. Each bar represents the fraction of a specific antibody mixture at a particular concentration combination, providing insights into the competitive dynamics and selection efficiency among antibodies with varying affinities. The use of long-read sequencing allows for a detailed and precise analysis of the complex interplay within these mixed phage populations.

### DETAILED DESCRIPTION

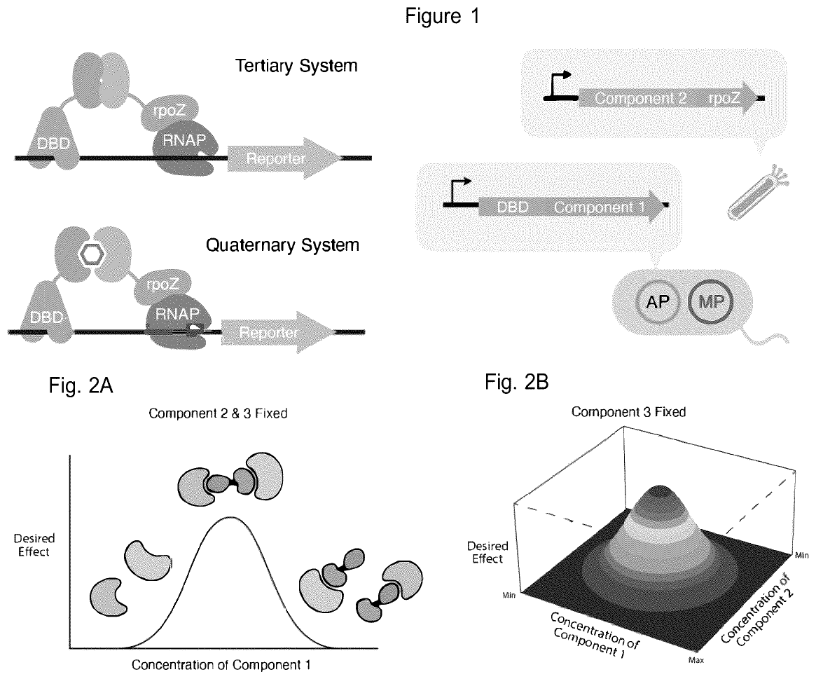

The invention, in part, includes systems and methods that significantly improve continuous evolution procedures for multi-body complexes and the quality and scope of results that can be obtained with such approaches. It has now been discovered that by optimizing evolution conditions in logarithmic continuous phage, it is possible to identify optimal conditions that will allow for favorable evolution of binding interactions, by being able to accurately titre the equilibrated protein levels using inducible promoters. Embodiments of methods of the invention can be used to optimize the concentrations of 3-body complexes or 4-body complexes in continuous logarithmic growth with effective dose titration, and it has now been identified that in the absence of such optimization, prior efforts were operating under conditions that favored binding outcomes with lower, rather than higher avidity for their targets. FIG. 1 illustrates a 3-molecule multi-body complex (tertiary system) and a 4-molecule multi-body complex (quaternary system).

The invention, in part provides methods with which to identify conditions that give rise to equimolar ratios of molecules capable of forming a multi-body complex of interest. In some embodiments, the evolution is not performed at equimolar ratios. For example, to improve a molecule by evolution one can start the evolution process with one or more of the molecules that form a multi-body complex of interest “below” their equimolar ratio, or one may want to make binding of a molecule in a multi-body complex of interest slightly worse, in which case one would initiate evolution with one or more of the molecules that form the multi-body complex of interest at a slightly higher ratio than the equimolar ratio. A central advantage of methods and systems of the invention is their use to identify where the equimolar ratio occurs; the conditions and parameters are that result in that equimolar ratio; and how the conditions and parameters can be changed to accomplish an evolution goal that is of interest. The conditions identified using methods of the invention under which molecules forming a multi-body complex of interest are evolved or the identified conditions can be adjusted to selectively alter the evolution outcome for a multi-body complex of interest.

The invention, in part, includes methods for identifying and implementing protein-protein binding evolutions in which each of the three components in a tertiary system, or four components in a quaternary system, are provided at equimolar ratios during continuous evolution. The invention, in part, also includes methods for altering identified optimal conditions for protein-protein binding evolutions in which each of the three components, or four components are not held at equimolar ratios during continuous evolution. Systems and methods of the invention can be used to identify conditions that result in equimolar ratios of components that form a multi-body complex of interest, which permits a user to adjust and alter the ratios of one or more of the components in a continuous evolution system to tailor the outcome of the molecule evolution. For example, identifying conditions that result in equimolar ratios of the component molecules comprising a multi-body complex and that can be altered to either decrease binding affinity or increase binding affinity in the evolved molecules of the multi-body complex. Thus, systems and methods of the invention add efficiency and flexibility to evolution of 3-component and 4-component multi-body complexes and permit production of highly effective molecules for therapeutics, research, etc.

### Hook Effect

Generally, previous continuous evolution systems used constitutive promoters to express the relevant components, because in continuous flow, the cells of interest are being cultured in growth equilibrium. However, it has now been determined that the impact of the hook-effect in 3-component and 4-component systems is so strong, that without effective dose-optimization, previous attempts at protein-interaction evolution generated proteins with lesser, rather than improved function. FIG. 2A-B illustrates impact of a “hook-effect” on multi-body complexes. Unlike in many previous protein evolution “circuits” that have been successfully implemented with continuous evolution (polymerases, synthetases, proteases, etc.), three-body component systems such as bacterial “2-Hybrid” and chemically induced dimerization are uniquely subject to a phenomenon known as the “hook-effect” (alternatively known as the “prozone effect”). The hook-effect is a kinetic principle which designates that in order to achieve the maximum desired effect, all three biomolecules (in the case of 2-hybrid) or the 2 biomolecules and small-molecule (in the case of chemical dimerization), must be provided in equimolar ratios. As such, there are three potential phases in which evolution may take place. The first condition (Regime 1) occurs when all components are provided in equimolar ratios, which is where the maximum effect is observed. In Regime 2, if the intermediate component is provided at too low of a level compared to the two proteins it is dimerizing, the effect observed is less than the possible maximum. Finally, if the intermediate component is provided at too high of a level compared to the proteins it is dimerizing, the effect observed is also less than the possible maximum (Regime 3). This phenomenon has broadly inhibited the optimal development of evolution “circuits” that rely on or seek to evolve protein-protein interactions. Similarly, the hook effect can occur in evolution circuits comprising a four-molecule multibody complex. Methods of the invention; can be used to reduce and/or eliminate the hook effect in evolving quaternary multi-body complexes.

### Sweet Spot Identification

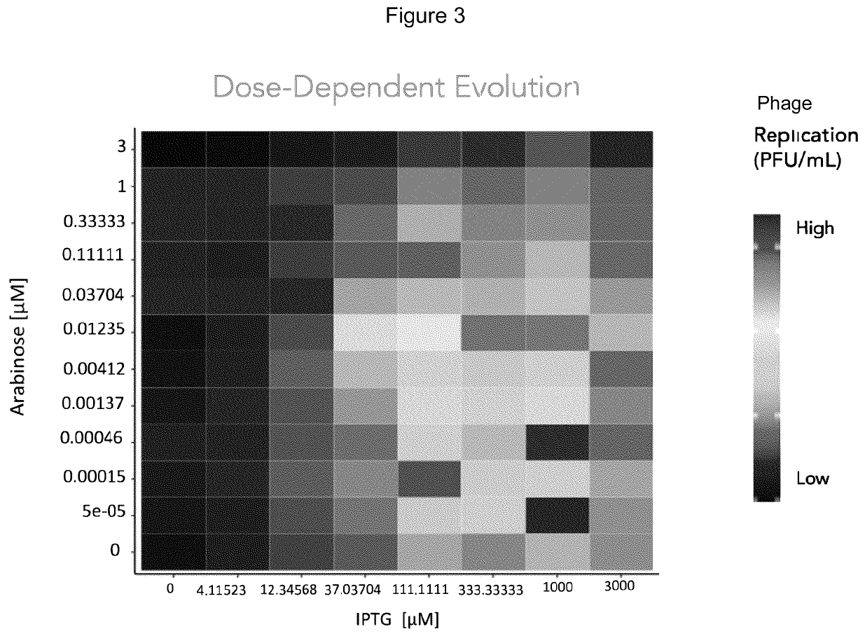

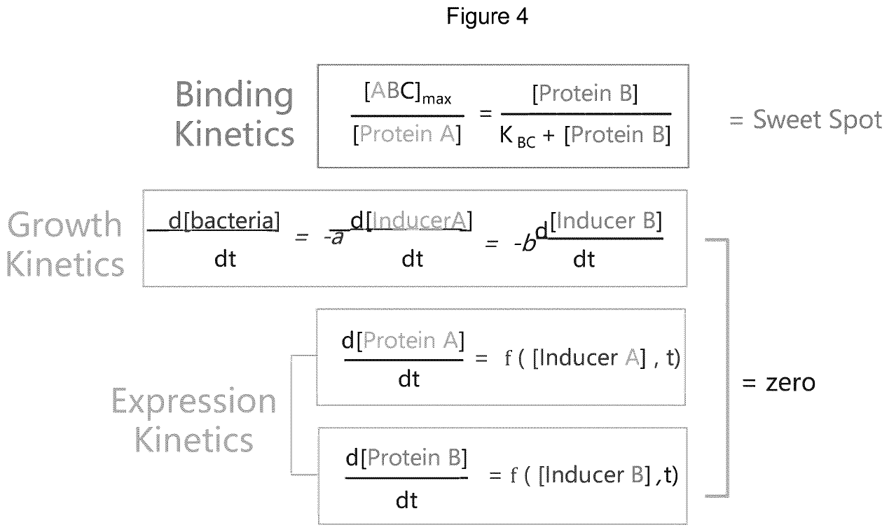

To identify and evolve proteins in multi-body complexes, it has now been discovered that by expressing the proteins of the multi-body complex in an evolution circuit under inducible promoters, and using an automated, high-throughput turbidostat method on a liquid handling robot to eliminate the growth and pre-equilibrium kinetics from protein induction, an optimized “sweet spot” can be identified that will most favor the improvement of protein-protein binding when coupled with protein mutagenesis and selection. As used herein the term “sweet spot” means referring to the optimum concentrations of components that form a multi-body complex that allow for the maximum amount of phage replication that corresponds with activity, without going above a concentration that starts to favor reverse evolution. For example, the ideal/max concentrations at which an evolution event can occur. FIG. 3 illustrates that any concentration that is greater than the local maximum will not favor evolution. FIG. 4 shows mathematical analysis of kinetics that result in obtaining the sweet spot in an evolution circuit and kinetics that do not.

In some embodiments of systems and methods of the invention, a robotic platform is used to establish both kinetically and growth-equilibrated conditions to improve or purposefully reduce the binding affinity of biomolecules of interest, a non-limiting example of which are molecules capable of forming a multi-body complex. In some embodiments of systems and methods of the invention an organism or cell that is being used for protein evolution of a multi-body complex of interest is cultured on a robotic liquid handling system in which the organism density is maintained by a feedback controller built into the liquid handling robot. Following equilibration of growth, a first molecule (also referred to herein as a first component) of the multi-body complex is then induced under a wide range of doses using a small molecule and allowed to reach kinetic equilibrium. The second and third molecules (in a 3-component multi-body complex) or the second, third, and fourth molecules (in a 4-component multi-body complex) are then induced, each allowing for kinetic equilibrium to be reached, before protein evolution through mutagenesis is initiated. Using a reporter for expression the “sweet spot” of the evolution circuit is then identified, which allows successful protein evolution to occur, even though the system is bivariate. In the absence of this equilibration system, it is not possible to identify if the conditions that are being employed during protein evolution are more or less likely to favor evolving improved or weakened protein avidity, as both would appear to give the same result in the absence of non-equilibrium optimization.

### Applications of Certain Methods

Non-limiting examples of applications of systems and methods of the invention to evolve multi-body complexes include evolution of molecules capable of forming multi-body complexes for use in biologic therapies with improved avidity against protein targets. The term “capable of forming” as used herein in reference to molecules forming a multi-body complex, refers to molecules that in certain methods of the invention form a multi-body complex. For example, embodiments of methods and systems of the invention can be used to prepare an evolution circuit comprising a plurality of molecules capable of forming a multi-body complex, and in some embodiments of methods and systems of the invention, a plurality of molecules capable of forming a multi-body complex form the multi-body complex. Additionally, one can use embodiments of systems and methods of the invention to generate protein mutations in known protein interactions that can be used to generate improved protein crystal structures, as many naturally protein interactions do not form strong enough binding interactions to favor protein crystallization.

Systems and methods of the invention may also be used to improve machine-learning data, which can be used to optimize continuous evolution of multi-body complexes. As a non-limiting example, embodiments of systems and methods of the invention can be used to generate large amounts of training data in the protein-binding sequence space, resulting in data that is representative of high-avidity interactions, as naturally occurring protein interactions generally were not evolved to favor high avidity. Use of machine learning data for binding activity trained on known protein interactions, in conjunction with systems and methods set forth herein, permits the user to both create large improvements or subtle binding differences. Some of the most potent therapeutic binding proteins, such as antibodies, are not remotely optimized for binding strength, because the immune system is not designed for the strongest binding interactions, as they often create negative effects during immune tolerance. Because systems and methods of the invention permit optimization of evolution of molecules of multi-body complexes to produce a desired level of binding strength of the molecules, embodiments of systems and methods of the invention can be used to optimize binding of antibodies and other therapeutic binding molecules.

Certain aspects of the invention include methods for optimizing the magnitude of expression to favor optimized binding between two proteins within a bacterial two-hybrid system, thereby allowing for evolution to occur below the point at which reverse evolution becomes favored over forward evolution. In some embodiments of methods of the invention, expression of a reporter, non-limiting examples of which are luminescence reporters, fluorescence reports, and phage protein reporters, is linked to a binding interaction between proteins in a multi-unit complex, also referred to herein as a multi-body complex. In some embodiments of methods of the invention comprise use of kinetic optimization methods to prepare and use novel protein binding circuits, and in the high-throughput determination of the mutation space that favors favorable or disfavorable protein binding mutations.

Certain embodiments of methods of the invention can be used to replace use of DNA screening libraries and alternative methods of directed evolution to enrich for proteins with improved binding to a protein of interest. Non-limiting examples of embodiments of the invention may include multi-body complexes comprising: (i) a DNA binding domain fused to the N-terminus of single chain antibody or nanobody; (ii) an antigen fused to the N-terminus of the rpoZ protein, which recruits (iii) a native DNA-directed RNA polymerase (RNAP) to a genetic region of interest upstream of a gene that acts as a i) reporter, ii) selectable marker iii) protein required for phage propagation.

In another non-limiting example, certain embodiments of the invention include multi-body complexes comprising: (i) a DNA binding domain fused to the N-terminus of a protein capable of binding a small molecule (ii), a second protein capable of binding a separate small molecule, (iii) a small molecule that is capable of chemically dimerizing with components (i) and (ii) to recruit (iv) a native RNAP to a genetic region of interest upstream of a gene that acts as a i) reporter, ii) selectable marker iii) protein required for phage propagation.

### Evolution Circuit

Methods of the invention include preparing and using an evolution circuit for directed evolution of molecules in multi-body complexes. As known in the art, an evolution circuit comprises genetic components, for example: promoters, genes, inducers, plasmid origin, etc. that are necessary to combine and use in a manner such that when all the components are functional, protein binding will result in phage replication, and subsequently increased protein binding will result in more rapid phage replication. Systems and methods of the invention include preparing an evolution circuit that is used to assess and determine conditions under which components of 3-molecule or 4-molecule multi-body complexes reach kinetic equilibrium. Conditions determined for the evolution circuit can then be maintained during evolution of the 3-molecule or 4-molecule multi-body complexes or, in some embodiments of systems and methods of the invention the determined conditions are altered in order to impact evolution of components of the 3-molecule or 4-molecule multi-body complexes, respectively.

Certain embodiments of a system or method of the invention provides an evolution circuit that is a 2-Hybrid circuit in which one protein within an interaction is expressed as a fusion to a DNA binding domain, while a second component of a protein interaction is expressed as a fusion to a transcriptional activator protein. In some embodiments of the invention, the DNA binding domain is expressed constitutively, while the activation domain is expressed inducibly and equilibrated on a liquid handling robot. In some embodiments of the invention, the activation domain is expressed constitutively, while the DNA binding domain is expressed inducibly and equilibrated on a liquid handling robot. In some embodiments of the invention, both the activation domain and DNA binding domain are expressed inducibly and equilibrated on a liquid handling robot. In some embodiments of the invention, one or either of the domains are expressed from bacteria, while the other component is expressed from bacteriophage. In some embodiments of the invention, both of the domains expressed from bacteriophage. In some embodiments, one or more small molecule inducer is added by the liquid handling robot in a dose-responsive manner to establish titration curves of small molecules involved in the system of the invention. In some embodiments, a DNA binding domain binds a specific sequence of DNA resulting from a protein-DNA interaction, in other embodiments of systems and methods of the invention, a DNA binding protein is a CRISPR protein, which may be recruited to a sequence of interest.

A non-limiting example of an evolution circuit of the invention comprises the following components: (1) the DNA binding protein+antigen (protein fusion) expressed under an inducible promoter (pLac, pBad, etc.), off of a plasmid; (2) the rpoZ protein (RNAP subunit)+antibody (protein fusion) expressed under a different inducible promoter (plac, pBad, etc.) off of a plasmid or in the genome; (3) RNAP expressed endogenously in bacteria; and (4) various inducer concentrations that control the strength of induction for the various promoters. When the above components are expressed in the right equimolar ratio, they result in the expression of a protein required for phage proliferation. This could either include: (1) all components result in the direct expression of the protein pIII, which is required for phage expression; (2) all components result in the expression of a T7 Polymerase which then drives the expression of pill which is required for phage expression; (3) all components result in the expression of one subunit of a split T7 polymerase, which when co-expressed with the other split subunit, drives the expression of pIII, which is required for phage expression. In all cases, pIII could be replaced with another required phage protein (such as pVI, pIV, etc., there are 9 total), or even in another phage. In addition, many variations of promoters/polymerases could be used in place of the T7RNAP, to control expression of any of the components.

Another non-limiting example of an evolution circuit of the invention comprises all possible permutations of promoter/antigen/antibody combinations, and also the various permutations of inducible RNAP that could also be expressed under inducible control.

Another non-limiting example of an evolution circuit of the invention comprises: (1) the DNA binding protein+surface receptor (protein fusion) expressed under an inducible promoter (pLac, pBad, etc.), off of a plasmid; (2) The rpoZ protein (RNAP subunit)+receptor ligand (protein fusion) expressed under a different inducible promoter (plac, pBad, etc.) off of a plasmid or in the genome; (3) RNAP expressed endogenously in bacteria; and (4) various inducer concentrations that control the strength of induction for the various promoters. Non-limiting examples of promoters that may be included in an evolution circuit of the invention are: pLAC (IPTG), pTet (tetracycline inducible), pBAD (arabinose inducible), jEDx (crystal violet inducible), pPSP (phage shock promoter. Other non-limiting examples of evolution circuits of the invention are: DBD-ProteinA+rpoZ-ProteinB+RNAP (under all possible promoter plasmids permutations).

### Multi-Body Complexes

Certain embodiments of methods of the invention can be used to evolve proteins capable of forming a multi-body complex. Such evolution in embodiments of methods and systems of the invention can be used to improve (or reduce) binding affinity of the proteins to their native targets, non-limiting examples of which are small molecules, protein-protein interactions (PPIs), and antigens. In some embodiments, the invention comprises a system or method of evolving molecules of multi-body complexes. The systems or methods may include (a) preparing an evolution circuit comprising a plurality of molecules capable of forming a multi-body complex; (b) forming the multi-body complex comprising the plurality of molecules in the prepared evolution circuit; (c) determining for each of the plurality of molecules, one or more equilibrium conditions in the prepared evolution circuit capable of producing an equimolar ratio of each of the plurality of molecules in the evolution circuit; (d) selecting one or more evolution conditions for the plurality of molecules based at least in part on the one or more equilibrium conditions determined for each of the plurality of molecules; (e) maintaining the selected one or more evolution conditions; and (f) evolving one or more of the molecules of the multibody complex under the maintained one or more selected evolution conditions.

As used herein, the term “plurality” in reference to a molecule of a multi-body complex means more than one. In some embodiments of methods and systems of the invention, the term “plurality of molecules capable of forming a multi-body complex” means three molecules capable of binding together thereby forming the multi-body complex. In this instance, the multi-body complex comprises the three molecules. In some embodiments of methods and systems of the invention, the term “plurality of molecules capable of forming a multi-body complex” means four molecules capable of binding together thereby of forming the multi-body complex. In this instance, the multi-body complex comprises the four molecules.

Many different 3- and 4-multibody complexes are known in the art and are suitable for continuous evolution using an embodiment of a system and/or method of the invention. In some embodiments, methods and systems of the invention are used to evolve one or more preselected gene of interest that encodes a region of a protein that is known to bind with a measurable affinity to a biomolecule of interest. For example, though not intended to be limiting, a preselected gene of interest may be a gene encoding a protein that binds with any genomically encoded biomolecule. Non-limiting examples of embodiments of systems and methods of the invention are their use to evolve a gene that encodes a region of one or more proteins involved in protein binding and/or interaction such as those between: an antigen and an antibody, a leucine zipper dimer, a surface receptor and a viral coat protein, a toll-like-receptor and cognate ligand, a signal transduction interaction (Ras/Raf), protein aggregates including Tau, and a surface protein and its ligand (PD-1/PDL-1).

In some embodiments, a plurality of molecules capable of forming a multi-body complex comprise one or more proteins and small molecules. Certain embodiments of systems and methods of the invention are used to evolve a protein-protein interaction that is mediated by the titration of a small molecule dimerizer. Non-limiting examples are protein complexes encoded by genes whose interactions are mediated by naturally occurring bifunctional small molecules non-limiting examples of which are Rapamycin, cyclosporine, FK506, abscisic acid, and gibberellin. A non-limiting example, in which the plurality of molecules capable of forming the multi-body complex comprises proteins and small molecules that dimerize with each other, is a multi-body complex comprising FKBP-rapamycin-Frb. Some embodiments of methods and systems of the invention are used to evolve a protein interaction that is mediated by the titration of non-naturally occurring bifunctional small molecules, non-limiting examples of which are FK1012, AP1903, FKCsA, and PROTACs.

In some embodiments, a plurality of molecules capable of forming the multi-body complex comprise proteins that dimerize with each other, non-limiting examples of such multi-body complexes are leucine zippers, alpha helical complexes, etc.). In some embodiments, a plurality of molecules capable of forming the multi-body complex comprises proteins chemically linked to a small molecule that binds an enzyme. In some embodiments, the plurality of molecules capable of forming the multi-body complex comprise proteins capable of recruiting other proteins, non-limiting examples of which are proteins that recruit other proteins via peptides/small molecules, such as but not limited to degraders. As used herein the term “degraders” refers to small molecules that bind two different proteins, in which one binds the protein of interest (to be degraded) and the other binds a protein that targets the 3-body complex to the proteosome for degradation (non-limiting examples of such molecules include pthalidomide and synthetic peptides that bind and recruit proteins to the proteome).

In some embodiments, the plurality of molecules capable of forming the multi-body complex comprise chemically modified proteins, which in some embodiments may be proteins chemically modified with small molecules that bind other proteins, non-limiting examples of which are binding via Click chemistry, post-translational modification, non-canonical amino acids, etc. As a non-limiting example: a first protein of a multi-body complex of interest is a DNA binding domain and is chemically modified such that it was fused to a small molecule that another protein bound tightly to. A non-limiting example of such is DBD modified with biotin (which would bind with rpoZ-streptavadin), or the DBD fused to a small molecule drug candidate (such as a kinase inhibitor), and then the rpoZ protein fused to a kinase. In each instance, when the molecules bind, they form a 3-molecule multi-body complex. In some embodiments of the invention, the molecules are not originally provided to the bacteria as such.

In some embodiments, the plurality of molecules capable of forming the multi-body complex comprise molecules capable of dimerizing with two proteins of a same kind, non-limiting examples of which are signaling proteins. In some embodiments the plurality of molecules capable of forming the multi-body complex comprise molecules capable of use in protein purification methods and can be expressed in or provided to cells, non-limiting examples of which are: streptavidin-biotin, halo-tag, etc. In some embodiments, the plurality of molecules capable of forming the multi-body complex comprise proteins capable of photo-cage induction, non-limiting examples of which are light-inducible dimers.

Certain embodiments of systems and methods of the invention, comprise evolving two or more preselected genes that encode proteins and the encoded proteins and/or two or more small molecules can form hetero or homodimerizing complexes. As a non-limiting example, two or more preselected genes that encode proteins are evolved and the proteins encoded by the evolved genes and/or two or more small molecules can form hetero or homodimerizing complexes. In certain embodiments of systems and methods of the invention a binding affinity between proteins created with de novo design that are capable of forming a multi-body complex, is evolved resulting in a change in a level of protein interaction between the molecules capable of forming the multi-body complex. In some embodiments, the level of protein interaction is improved versus an initial level and in certain embodiments, the level of protein interaction is purposefully reduced versus the initial level of protein interaction. A non-limiting example of a de novo-designed protein interaction of interest includes engineered nanobodies or coiled-coil domains.

In some embodiments of a method of the invention, one or more of the molecules that form a multi-body complex of interest is a protein and preparing the evolution circuit comprises expressing the one or more proteins in a cultured organism and/or a cultured cell. In some embodiments, two of the molecules capable of forming the multi-body complex are inducible proteins. In certain embodiments, the two (or more) inducible proteins are expressed in plasmids in a cultured cell. In some embodiments, one of the proteins is an exogenously expressed first protein fused to a DNA binding domain. In some embodiments, one of the proteins is an exogenously expressed second protein fused to a DNA-directed RNA polymerase (RNAP) subunit. In some embodiments, the one of the molecules capable of forming the multi-body complex is a DNA-directed RNA polymerase (RNAP) expressed by the cultured cell, and in certain embodiments, the RNAP expressed by the cultured cell is endogenous to the cultured cell. In some embodiments, the method also includes contacting the cultured organism in the evolution circuit with one or more independently selected inducible promoter to induce expression of the one of more of the proteins of the multi-body complex. Following expression of the one or more of the proteins of the multi-body complex, certain embodiments of methods of the invention also include determining an amount of the first, second, and third molecules in the evolution circuit, wherein a means of determining the amount comprises including at least one independently selected detectable label on one or more of the first, second, and third molecules. In a non-limiting example, in an embodiment of a method of the invention, the first molecule of a three-molecule multi-body complex is an exogenously expressed first protein fused to a DNA binding domain; the second molecule of the three-molecule multi-body complex is an exogenously expressed second protein fused to a DNA-directed RNA polymerase (RNAP) subunit; and the third molecule of the three-molecule multi-body complex is a molecule capable of dimerizing the first and second molecules of the three-molecule multi-body complex.

It will be understood that methods and systems of the invention can also be used in evolution methods for four-molecule multi-body complexes. In some embodiments, the plurality of molecules is four molecules and a first molecule and second molecule of the molecules capable of forming the multi-body complex are inducible proteins. In some embodiments, the inducible proteins are expressed in plasmids in a cultured organism. In some embodiments, the first molecule is an exogenously expressed first protein fused to a DNA binding domain. In some embodiments, the second molecule is an exogenously expressed second protein fused to a DNA-directed RNA polymerase (RNAP) subunit. In some embodiments, the third molecule of the molecules capable of forming the four-molecule multi-body complex is a molecule capable of dimerizing the first and second molecules capable of forming the multi-body complex. In some embodiments, a fourth molecule of the molecules capable of forming the multi-body complex is a DNA-directed RNA polymerase (RNAP) expressed by the cultured organism, and in some embodiments, the RNAP expressed by the cultured organism is endogenous to the cultured organism.

### Inducing Agents and Promoters

The terms “inducing agent” or “inducer” as used herein mean an agent that when included in an evolution circuit of the invention induces expression of one of the molecules capable of forming the 3-molecule multi-body complex of interest, or one of the molecules capable of forming the 4-molecule multi-body complex of interest. In some embodiments of systems and methods of the invention, an inducing agent is a small molecule inducing agent. In certain embodiments, a small molecule-inducing agent is a promoter. In some embodiments of methods and systems of the invention, an inducing agent is light, non-limiting examples of which are inducing promoters, or proteins that dimerize upon the addition of light.

Certain methods of the invention replace the use of constitutive promoters with chemically inducible promoters or inducible agents that allow for fine titration of protein expression. Non-limiting examples of inducible promoters that may be used in methods of the invention are pBAD, pLac, pJExD, pTet, pVan. Additional non-limiting examples of inducible promoters that may be used in certain embodiments of systems and methods of the invention are environment-specific promoters (for example, but not limited to heat shock, phage shock, quorum-sensing promoters etc.). It will be understood that a promoter may be selected and used in systems and methods of the invention if it can be induced by or upon some trigger compatible with the system and kinetic control can be used to fine-tune the dose.

In some embodiments of methods of the invention, continuous flow is used to optimize conditions that enable sequential titration of one or more inducible promoters to optimize for leaky expression, which is prohibitive in continuous evolution using bacteriophage. In some embodiments of methods of the invention one or more inducible genes are expressed from bacteria, and in other embodiments of methods of the invention, one or more inducible genes are encoded in the genome of bacteriophage. In certain embodiments of systems and methods of the invention, one or more promoters is a constitutive promoter.

Some embodiments of systems and methods of the invention include methods of evolving biomolecules across a large a plurality of different conditions and combinations of conditions to generate datasets linking genotype to improved or weakened binding affinity for machine learning. Certain embodiments of systems and methods of the invention include methods of evolving native protein-protein interactions and identifying mutations in one or more of the interacting proteins that result in more favorable protein crystallization conditions for protein structural studies.

### Cells

As described herein, a cell used in a system or method of the invention may be an in vitro cell in which the cell is growing continuously in logarithmic phase. Such cells are also referred to herein as “cultured cells” (or “cultured organisms” in the case of bacteria and yeast). Non-limiting examples of types of cells that can be used in embodiments and methods of the invention are prokaryotic cells, for example bacterial cells. In some embodiments, a cell used in a system or method of the invention is a bacterial cell. In some embodiments of systems and methods of the invention, the bacterial cell comprises a bacteriophage, which is also referred to herein as “bacteria+phage”. A further non-limiting example of a type of cell that may be used in certain embodiments of systems and methods of the invention is a eukaryotic cell, non-limiting examples of which are a yeast cell, a mammalian cell, a non-human mammalian cell, or an insect cell. In some embodiments, a cell used in system or method of the invention is a cultured cell. A cell used in some embodiments of systems and methods of the invention is a cell that is growing continuously at log phase and undergoing evolution with a cognate virus.

In some embodiments, a cell that may be used in methods and systems of the invention are of a bacterial strain in which the rpoZ subunit has been deleted (delta-rpoZ). A non-limiting example is a bacterial strain available on addgene that is commonly used (see www.addgene.org/105064/), but most of the standard E. coli strains are compatible, as long as they contain the rpoZ deletion, or are strains in which the rpoZ protein can be deleted. The standard genetic modifications to the strains that may be used in methods and systems of the invention may include the over-expression of the proteins required for inducible protein control (tetR, LacI, etc.).

The systems and methods discussed herein may be used to evolve one or more molecules that form 3-molecule or 4-molecule multi-body complexes using bacteria+phage, yeast, or other cell type suitable for evolution. One of ordinary skill in the art will appreciate that in another embodiment, other sample materials and liquid exchange fluids may be used. In certain embodiments, samples may comprise three molecules capable of forming a three-molecule multi-body complex that is of interest for evolving in the evolution circuit, or samples may comprise four molecules capable of forming a four-molecule multi-body complex of interest for evolving in the evolution circuit. In some embodiments, cells may comprise a gene or genes encoding one or more molecules of interest to evolve in a 3-molecule multi-body complex or one or more molecules of interest to evolve in a 4-molecule multi-body complex.

One of ordinary skill in the art will appreciate that the systems and methods discussed herein may be used to evolve one or more molecules that can be coded and expressed that can form a 3-molecule multi-body complex of interest or to evolve one or more molecules that can be coded and expressed that can form a 4-molecule multi-body complex of interest.

Cells included in certain embodiments of systems and methods of the invention may be interchangeably referred to herein as cells or organisms (in the case of bacterial and yeast). In certain embodiments of the invention, a cell is a bacterial cell, non-limiting example of which is a bacterial cell+phage, (a bacterial cell infected with a phage). In some embodiments, a cell used in an evolution circuit of the invention is a yeast cell. Additional information about and examples of bacteria suitable for use in methods and systems of the invention are known in the art, see for example, U.S. Pat. No. 9,394,537, and Chory, E. et al., Mol Syst Biol 2021 March; 17(3): e9942. doi: 10.15252/msb.20209942, the content of each of which is incorporated by reference herein in its entirety.

### Certain Embodiments of Systems and Methods

Certain embodiments of systems and methods of the invention may include performing a plurality of parallel instances of the same evolution experiment in order to explore a variety of experimental conditions and inducer concentrations and to utilize real-time monitoring and feedback control over experimental conditions. Embodiments of systems and methods of the invention are used to determine conditions suitable to perform successful molecular evolution of molecules that form multi-body complexes. Embodiments of systems and methods of the invention permit flexibility in determining and producing, and optimizing conditions for continuous evolution of one or more molecules capable of forming a multi-body complex. Embodiments of systems and methods of the invention can be used to determine conditions for continuous evolution of one or more molecules that can form a 3-molecule multi-body complex. Embodiments of systems and methods of the invention can be used to determine conditions for continuous evolution of one or more molecules that can form 4-molecule multi-body complex.

As set forth in more detail elsewhere herein, certain embodiments of systems and methods of the invention are used to conduct continuous evolution of one or more molecules capable of forming a multi-body complex, and thus evolving one or more molecules that in some embodiments of methods and systems of the invention form the multi-body complex. Certain embodiments of systems and methods of the invention may be configured to run in a multi-well plate format using a liquid handling robot. In some embodiments, the liquid-handling robot used in a system or method of the invention is configured to run up to hundreds or thousands of separate populations of a cell, (non-limiting examples of which are a population of bacteria+phage, a population of yeast, a population of a eukaryotic cell, etc.), in a simultaneous manner; customizing the conditions in each experiment; and monitoring the fitness of each population.

As described herein, systems and methods of the invention can be used to evolve molecules of multi-body complexes in a manner that optimizes the evolution to reach a desired level of binding of the evolved molecules of the multibody complex. The systems or methods may include (a) preparing an evolution circuit comprising a plurality of molecules capable of forming a multi-body complex; (b) forming the multi-body complex comprising the plurality of molecules in the prepared evolution circuit; (c) determining for each of the plurality of molecules, one or more equilibrium conditions in the prepared evolution circuit capable of producing an equimolar ratio of each of the plurality of molecules in the evolution circuit; (d) selecting one or more evolution conditions for the plurality of molecules based at least in part on the one or more equilibrium conditions determined for each of the plurality of molecules; (e) maintaining the selected one or more evolution conditions; and (f) evolving one or more of the molecules of the multibody complex under the maintained one or more selected evolution conditions. In some embodiments, the selected one or more evolution conditions are the same as the one or more determined equilibrium condition. In certain embodiments, the selected one or more evolution conditions are different from the one or more determined equilibrium conditions. Identification of conditions that result in an equimolar ratio of each of the plurality of molecules permits a user to alter the conditions in order to alter binding affinity of molecules evolved in the evolution circuit. For example, some embodiments, the one or more evolution conditions are selected that result in improved binding affinity of the molecules of the multi-body complex. In another non-limiting example, in some embodiments the one or more evolution conditions may be selected so they result in a reduced binding affinity of the evolved molecules of the multi-body complex.

In some embodiments of systems and methods of the invention, the evolution circuit is prepared with a liquid-handling robot, and the evolution of the molecules is carried out using the liquid-handling robot. Examples of liquid handling robots are known in the art and routinely used in molecule evolution methods. In some embodiments of systems and methods of the invention, the evolution circuit is prepared using an automated, high-throughput turbidostat method on a liquid-handling robot. Certain embodiments of systems and methods of the invention include growing (also referred to as culturing) an organism or cell selected because the organism or cell can be used in protein evolution in the automated, high-throughput turbidostat on the liquid-handling robot. Non-limiting examples of organisms and/or cells that can be cultured and used in systems and methods of the invention are bacteria+phage or yeast. The use of an automated, high-throughput turbidostat on the liquid-handling robot permits culturing and maintaining a selected density of the cultured cells. In some embodiments, the selected density of the cultured cells is maintained using a feedback controller in the liquid-handling robot.

Certain embodiments of methods and systems of the invention in which three-molecule multi-body complexes are evolved, further comprise equilibrating growth of the cultured cells and then optimizing evolution conditions of molecules in the multi-body complex. In some embodiments, systems and methods of the invention comprise contacting a plurality of portions of an equilibrated cell culture with one or more combinations of concentrations of a first-molecule inducing agent and a second molecule-inducing agent; inducing production of the first molecule of the multi-body complex and the second molecule of the multi-body complex; and identifying one or more combinations of concentrations of the first-molecule-inducing agent and the second molecule-inducing agent that result in kinetic equilibrium of the first molecule and second molecules in the evolution circuit. Following determination of conditions (e.g. concentrations, etc.) in the evolution circuit that result in kinetic equilibrium of the first and second molecules, a portion of the equilibrated cell culture comprising one of the identified combinations of concentrations of the first-molecule-inducing agent and the second molecule-inducing agent that results in kinetic equilibrium of the first molecule and second molecules in the evolution circuit may be contacted with a predetermined concentration of the third molecule of the multi-body complex that results in kinetic equilibrium of the third molecule. The kinetic equilibrium of the first, second, and third molecules may be maintained and evolution initiated, or the kinetic equilibrium may be altered by shifting one of more of the predetermined concentrations of the first, second, and/or third molecule to either decrease binding affinity between two or more of the first, second, and third molecules of the multi-body complex, or to increase binding affinity between two or more of the first, second, and third molecules of the multi-body complex. In some embodiments, the cultured cell is a bacterium, and the method further comprises contacting the cultured cell comprising the first, second, and third molecules of the multi-body complex maintained at kinetic equilibrium with a phage. In certain embodiments, initiating evolution comprises initiating mutagenesis in the cultured cells in the evolution circuit when the first, second, and third molecule are at their respective kinetic equilibriums. In certain embodiments, initiating evolution comprises initiating mutagenesis in the cultured cells in the evolution circuit when the each of the first, second, and third molecules of the multi-body complex is not maintained at its kinetic equilibrium.

In some embodiments a method of the invention comprises determining in an evolution circuit an amount of a first, second, and third molecule capable of forming a 3-molecule multi-body complex of interest, wherein a means of determining the amount comprises including at least one independently selected detectable label on one or more of the first, second, and third molecules. In some embodiments, a method of the invention comprises identifying of the one or more conditions in an evolution circuit that result in kinetic equilibrium of the first, second, and third molecules. In some embodiments, the identification method comprises contacting a cultured cell with a plurality of amounts of a first molecule-inducing agent and second molecule-inducing agent, respectively; determining an amount of the first molecule-inducing agent that induces the kinetic equilibrium of the first molecule; and determining an amount of the second molecule-inducing agent that induces the kinetic equilibrium of the second molecules. Conditions in the evolution circuit that determine the presence of kinetic equilibrium of a molecule include, but are not limited to an amount of the inducing agent contacting a cultured cell in the evolution circuit, identity of the inducing agent, timing of an initial contact of the cultured cell with the inducing agent, and a length of time of contact of the cultured cell with the inducing agent. Additional non-limiting examples of condition that can be identified and adjusted in certain embodiments of methods of the invention include one or both of the length of time and the order of timing events. For example though not intended to be limiting, one molecule capable of forming a multi-body complex might require an identified amount of time to reach kinetic equilibrium, but the time the inducing agent for that molecule is added to the evolution circuit can vary, for example, it may be added before the phage are added and the bacteria are equilibrating, or at the same time phage are added, or at another time determined to result in kinetic equilibrium of that molecule. In some embodiments, a produced kinetic equilibrium of the plurality of molecules results in an equimolar ratio of the plurality of molecules capable of forming the multi-body complex.

### General Overview of System Components

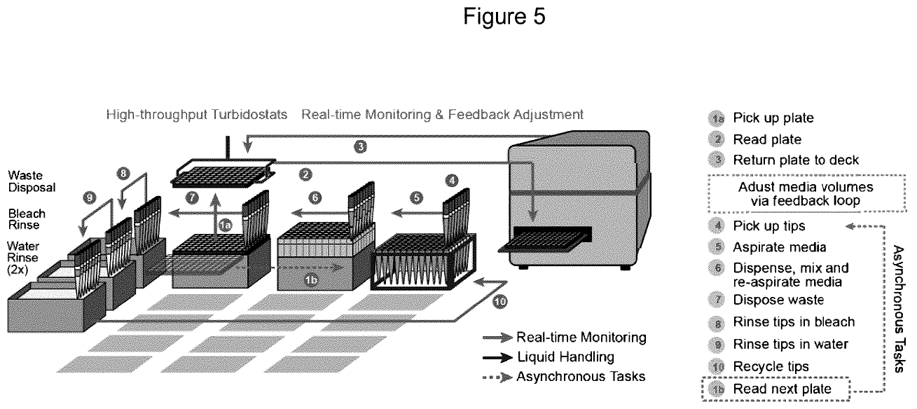

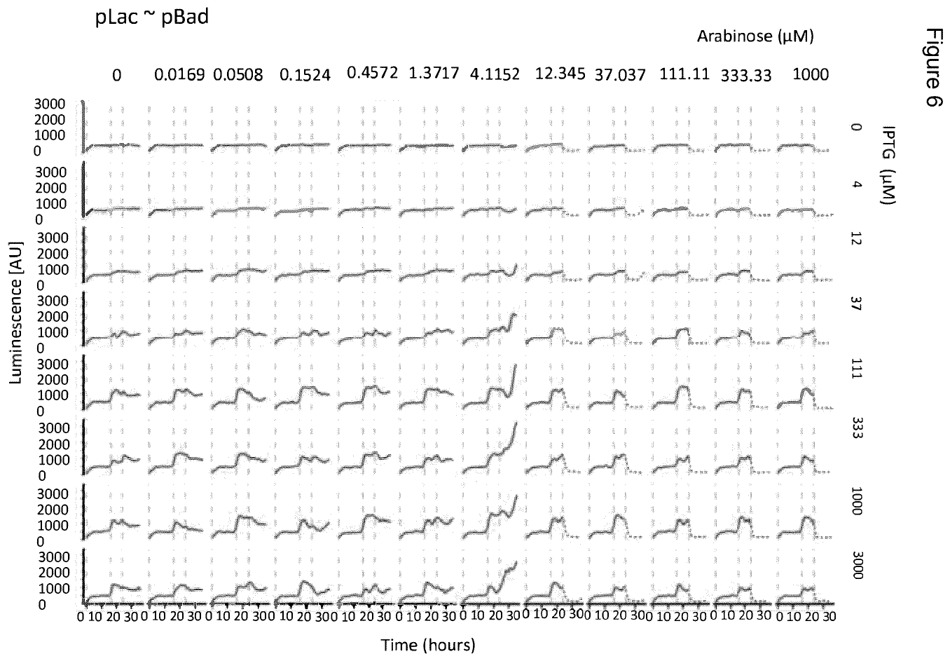

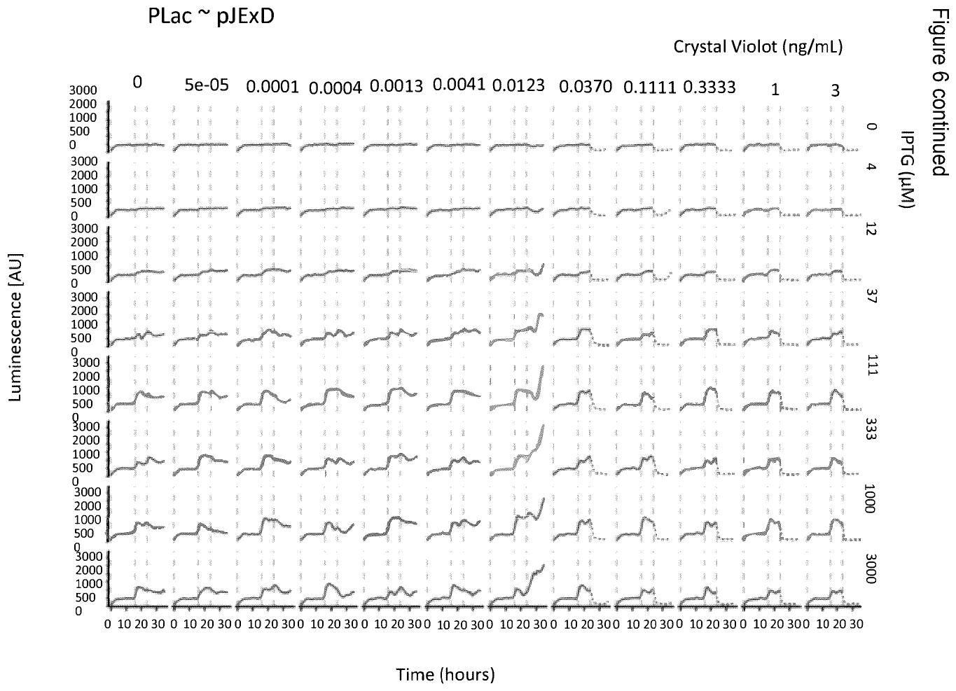

Systems of the invention comprise various components, non-limiting examples of which include one or more: receptacles, liquid-handling robots, sensors, integrated detectors, feedback controllers, non-integrated detectors, temperature controller elements, reservoirs, bacteriophage solutions, bacteria solutions, yeast solutions, inducing agents, reservoir ports, fluid lines, peristaltic pumps, sets of receptacles, detection receptacles, samples, cells, host cells, and transferrable nucleic acids. Information on various components is provided herein and it will be understood that art-known components of traditional continuous evolution methods and procedures may also be utilized in embodiments of systems and methods of the invention. A non-limiting example of a robotic system of the invention is shown in FIG. 5.

Robotic evolution procedures, components, and methods are known in the art and may be used in conjunction with the disclosure herein in methods and systems of the invention. See for example, references cited above and U.S. Pat. Nos. 9,394,537, 9,771,574; US Publication No. 2019/0345487, and Dickson, B. C. et al. Nat. Comm. Oct. 20, 2014, DOI:10.1038/ncomms6352, DeBenedictis, F. et al., Nat Methods, 2022 January; 19(1):55-64. doi: 10.1038/s41592-021-01348-4. Epub Dec. 30, 2021; and Chory, E. et al., Mol Syst Biol 2021 March; 17(3): e9942. doi: 10.15252/msb.20209942, the content of each of which is incorporated by reference herein in its entirety. Aspects of the present disclosure are directed to systems, components, and methods for conducting directed evolution that may be applied to problems previously intractable using traditional technology.

### Liquid Exchange and Handling

Another component included in embodiments of a system and method of the invention is a liquid handling robot. Although the physical size, shape, etc. may differ in different embodiments of the invention, a liquid handling robot is configured to perform liquid exchange in one or more receptacles in the plurality of receptacles. A liquid handling robot used in a system or method of the invention may be configured to add material to a receptacle, remove material from a receptacle, mix the contents of a receptacle, remove an aliquot of a sample from a receptacle, as well as for other tasks. As used herein, the term “material” used in reference to a receptacle is used to refer to a sample, a portion of a sample, an aliquot, liquid, a solution, a suspension, cells, or other suitable inclusion in a receptacle or set of receptacles of the invention. Liquid-handling robots are known and used in the art, see for example, DeBenedictis, E. et al., Nat Methods, 2022 January; 19(1):55-64. doi: 10.1038/s41592-021-01348-4. Epub Dec. 30, 2021; and Chory, E. et al., Mol Syst Biol 2021 March; 17(3): e9942. doi: 10.15252/msb.20209942, the contents of each of which is incorporated herein by reference in its entirety. It will be understood that alternative liquid handling robots suitable for use in systems and methods of the invention are commercially available or can be individually prepared for use in a system or method of the invention.

A liquid handling robot suitable for use in embodiments of systems and methods of the invention comprises one or more reservoirs, wherein each reservoir is configured to hold an independently selected fluid. A fluid in a reservoir of the invention may be a “liquid exchange fluid,” in that it is configured for one or more of: being added to and being mixed with one or more samples in a plurality of receptacles. A fluid in a reservoir may comprise bacteriophage and be referred to herein as a “bacteriophage solution.” A liquid-handling robot may be configured to selectively dispense a liquid, a non-limiting example of which is a bacteriophage solution, into one or more receptacle in a system of the invention. It will be understood that one or more of the receptacles into which a bacteriophage solution is added using a liquid handling robot of the invention may also include other materials. For example, in some embodiments of the invention a receptacle comprises one or more of an additional: liquid, solution, suspension, chemical, nucleic acid, polypeptide, and compound before or after addition of a bacteriophage solution by the liquid-handling robot of the invention.

Another non-limiting example of an independently selected liquid-exchange fluid that can be included in one or more liquid-handling robot reservoirs is a fluid comprising one or more inducing agents. It will be understood that an inducing agent or inducer, is an agent and/or small molecule that increases the rate of a gene mutation. Thus, in some embodiments of the invention, a host cell population may be contacted with an inducer for an inducible promoter in an amount sufficient to result in an increased rate of mutagenesis. As a non-limiting example, a bacterial host cell population is provided in which the host cells comprise a mutagenesis plasmid that includes an expression cassette and the expression cassette is controlled by an inducible promoter. Thus, when the population of host cells is contacted with the inducer, it induces an increased rate of mutation.

It will be understood that in some aspects of systems of the invention, one or more operations carried out by a system robot may be controlled with respect to timing, frequency and other manipulation parameters using software, a non-limiting example of which is scheduling software. It will be understood by those in the art how to select, design, and/or implement scheduling software in an embodiment of a system or method of the invention.

### Sensors and Detection

Another component that may be included in certain embodiments of systems and methods of the invention is a sensor configured to detect at least one detectable parameter of one or more of samples that are components in a system and/or method of the invention. In some aspects of the invention, one or more independently selected sensors are included. Non-limiting examples of a sensor that may be included in a system or method of the invention is a sensor configured to detect one or more of absorbance, luminescence, fluorescence, and color in one or more samples. A sensor included in an embodiment of the invention may be constructed and arranged as an integrated plate reader. It will be understood that alternative sensors are suitable for use in systems and methods of the invention, and are commercially available, or can be individually prepared for use in a system or method of the invention. In some aspects of the invention a sensor is a plate reader, which may be a plate reader that is integrated as part of the system of the invention or may be a plate reader that is a component separate from the system to which sample aliquots may be delivered for detection.

Timing and duration of detection of one or more samples can be independently adjusted and determined for one or more samples in some embodiments of systems and methods of the invention. As a non-limiting example, a sensor used in a system of the invention may be configured to detect over a predetermined time period of at least 1, 3, 6, 12, 18, 24, 36, 48, or more hours. Another detection element that can be adjusted for use in a system and/or method of the invention are intervals of detection. For example, a detector may be configured to detect one or more samples using one or more of the following parameters: (i) continuous detection over a period of time, (ii) interval detection over a period of time, and (iii) a combination of (i) and (ii) as desired by the operator of the system or method of the invention.

### Feedback Aspects

Some embodiments of systems and methods of the invention comprise a feedback controller component that may be configured to make one or more real-time adjustments in one or more samples in the evolution circuit. A feedback controller is used in some embodiments of systems and/or methods of the invention as a monitor that receives information about a sample from a sensor, determines whether or not an action is needed with respect to the sample, and triggers one or more actions if deemed necessary. A feedback controller of the invention is configured to utilize ongoing data collection by sensor(s) about one or more samples, and to instigate one or more adjustments in one or more samples in the evolution circuit. For example, though not intended to be limiting, density information is obtained for a sample by detecting a direct detectable parameter in a portion of cultured cells or organism and as a result of that information a feedback controller instigates an action that utilized a fluid-exchange robot of the invention to deliver an inducing agent to one or more samples, thereby adjusting the one or more samples. It will be understood that a detectable parameter identified in a detected sample may instigate one or more alterations in (i) only the detected sample; (ii) one or more samples but not the detected sample, and (iii) the detected sample and one or more additional samples.

A feedback controller included in a system and/or method of the invention permits real-time adjustment of a condition in one or more samples that are in a plurality of receptacles or one or more sets of receptacles. Non-limiting examples of real-time adjustments that can be instigated by a feedback controller in a system and/or method of the invention comprises modifying at least one of a: (a) fluid exchange frequency, (b) fluid exchange volume, (c) sample composition, (d) selection parameter, (f) selection stringency, and (g) selection goal in at least one of the samples in the plurality of receptacles. In some embodiments of systems and methods of the invention, one or more of samples in a plurality of receptacles comprise a population of bacteria+phage or yeast and a real-time adjustment comprises at least one of a: (a) fluid exchange frequency, (b) fluid exchange volume, (c) sample composition, (d) selection parameter, (f) selection stringency, and (g) selection goal in at least one of the samples.

### Reporters

Reporters used in certain embodiments of systems and methods of the invention comprise molecules that can be detected using one or more of: direct detection and indirect detection of an emission such as, but not limited to: absorbance, luminescence, and fluorescence. Detection of one or more reporters can provide information about an effect of one or more conditions in an evolution circuit, thus, reporters can be used to help optimize conditions for evolution of molecules of 3-molecule or 4-molecule multi-body complexes. Detecting a reporter may comprise determining one or more of a presence, an absence, and an amount of one or more detectable aspect of a reporter, such as absorbance, luminescence, and fluorescence. Other parameters may also be detected in certain embodiments of systems and methods of the invention and additional suitable parameters are known in the art.

Systems and methods of the invention permit flexibility in detection strategies. For example, detection can be independently performed on different samples in the system and/or method of the invention, which permits detection of one sample, a portion of all samples, or all samples in a continuous evolution procedure. A system of the invention may also be configured such that each sample is detected using an independently selected means, time, and schedule of detection. In some embodiments of the invention, a means for the determining of one or more conditions in the prepared evolution circuit capable of producing kinetic equilibrium of each of the plurality of molecules comprises detecting a detectable reporter.

### Additional Terms

As used herein the term “plurality” used herein in reference to cells, samples is always more than one, and in certain embodiments of the invention can be one or more of: at least 2, 3, 4, 5, 6, 7, 8, 9, 10, 11, 12, 13, 14, 15, 16, 17, 18, 19, 20, 25, 30, 40, 50, 60, 70, 80, 90, 100, 200, 300, 400, 500, 600, 700, 800, 900, 1000, 2000, 3000, 4000, 5000, 6000, 7000, 8000, 9000, 10,000 or more cells or samples, respectively, including every integer between each listed number. The terms “a cultured cell” or “the cultured cell” as used herein in reference to systems and methods of the invention, refers to a plurality of the cell.

As used herein the term “portion” means “part of” when used in reference to items and sets of items, such as but not limited to cells, cultured cells, and samples. Thus, as used herein, the term “portion of the equilibrated cell culture” means part of the equilibrated cell culture, which will be understood to contain a plurality of cells.

As used herein, the terms “nucleic acid” and “polynucleotide” are used interchangeably, and understood to include one or more of natural nucleosides, nucleoside analogs, and other variant nucleosides many of which are known and routinely used in the art. The terms “polypeptide” and “protein” are used interchangeably herein and may refer to one or more of: protein complexes, natural polypeptides, synthetic polypeptides, and recombinant polypeptides. Polypeptides used in embodiments the invention may comprise one or more of: a natural amino acid, a non-natural amino acid, a non-canonical amino acid, an amino acid analog, and other known variants. Structure, function, and use of variants of polypeptides are well known in the art. See for example, U.S. Pat. No. 9,394,537, which is incorporated by reference herein in its entirety.

The term “promoter” is used herein in reference to a nucleic acid molecule with a sequence recognized by the cellular transcription machinery and that is capable of initiating transcription of a downstream gene. A promoter may be constitutively active, meaning that the promoter is always active in a given cellular context, or it may be conditionally active, meaning that the promoter is only active in the presence of a specific condition. For example, a conditional promoter may only be active in the presence of a specific protein that connects a protein associated with a regulatory element in the promoter to the basic transcriptional machinery, or only in the absence of an inhibitory molecule. A non-limiting example of a conditionally active promoter is an inducible promoter that is active only in the presence of an “inducer” or “inducing agent”. A variety of constitutive, conditional, and inducible promoters are well known and routinely used in the art and one skilled in the art will be able to identify and use suitable promoters, including inducible promoters in embodiments of systems and methods of the invention.

### EXAMPLES

### Example 1

Evolution of a scFv and antigen interaction (FIG. 1) that is subject to hook effects (FIG. 2).

### Materials and Methods

Methods of the invention are used to identify equilibrated kinetic inducer conditions, using a liquid handling as described in Chory, E. et al., Mol Syst Biol 2021 March; 17(3): e9942. doi: 10.15252/msb.20209942, by equilibrating growth on the liquid handling robot, and then equilibrating expression, following induction with a small molecule.

### Results

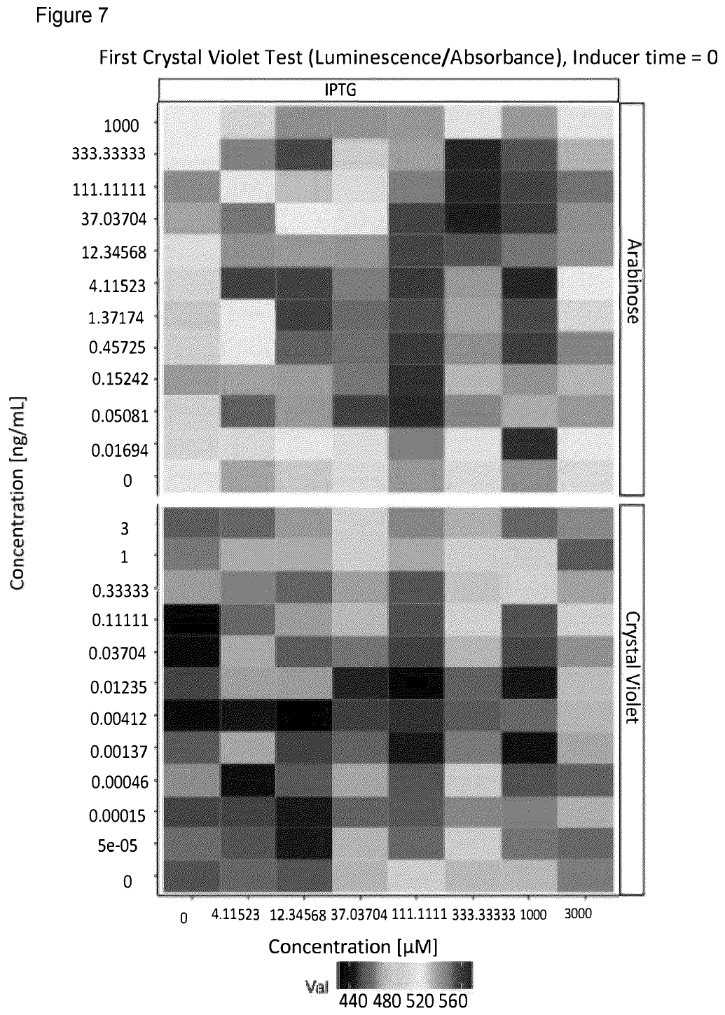

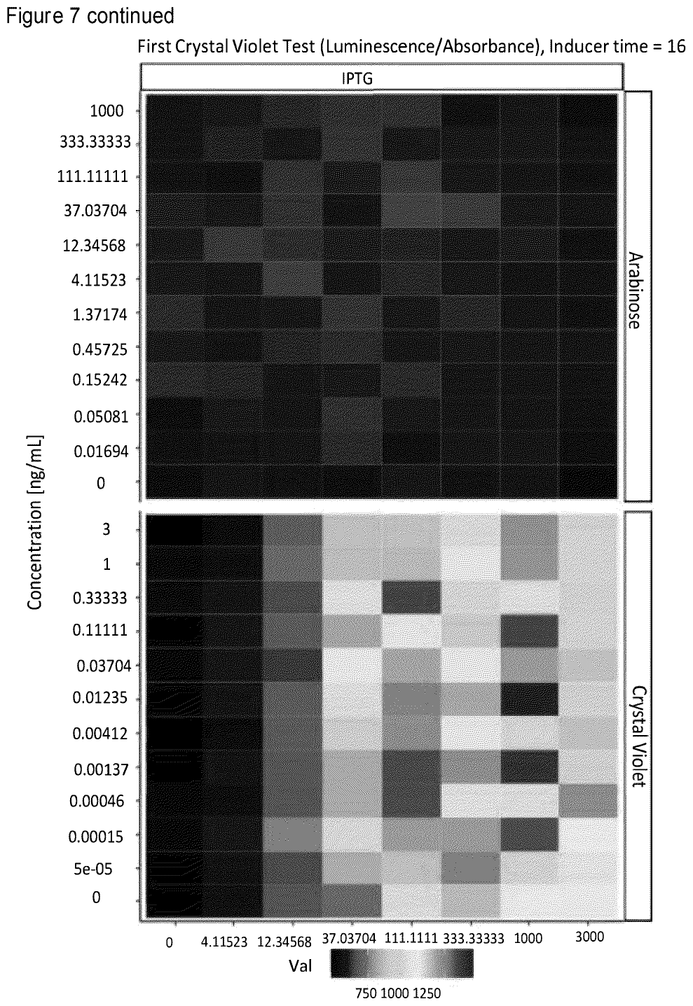

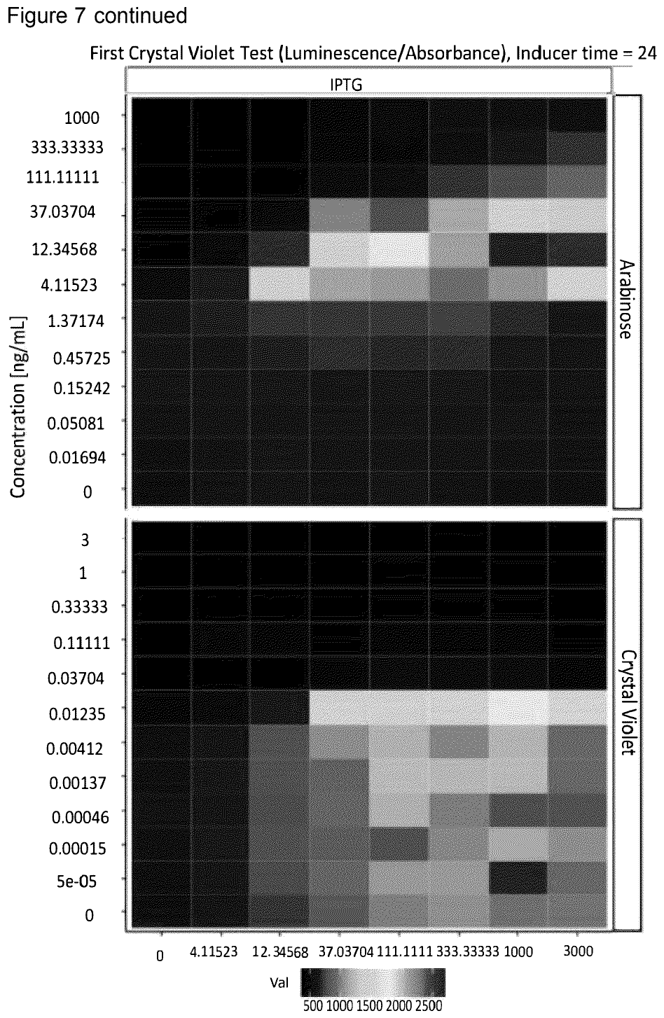

A “sweet spot” of protein binding in the experiment is identified, and protein evolution is performed in continuous flow using a combination of small molecules that favor improved binding (less than the maximum dose effect) (FIGS. 6 and 7).

### Example 2

Methods of the invention are used to identify equilibrated kinetic inducer conditions, using a liquid handling robot as described in Chory, E. et al., Mol Syst Biol 2021 March; 17(3): e9942. doi: 10.15252/msb.20209942, by equilibrating growth on the liquid handling robot, and then equilibrating expression, following induction.

In certain experiments, a plurality of molecules capable of forming a multi-body complex comprise one or more proteins and small molecules and methods described above herein are used to evolve a protein-protein interaction that is mediated by the titration of a small molecule dimerizer.

In some experiments, multi-body complexes are protein complexes encoded by genes whose interactions are mediated by naturally occurring bifunctional small molecules non-limiting examples of which are Rapamycin, cyclosporine, FK506, abscisic acid, and gibberellin.

In some experiments the plurality of molecules capable of forming the multi-body complex comprises proteins and small molecules that dimerize with each other, is a multi-body complex comprising FKBP-rapamycin-Frb.

In some experiments protein interaction in the evolution circuit is mediated by the titration of non-naturally occurring bifunctional small molecules, non-limiting examples of which are FK1012, AP1903, FKCsA, and PROTACs.

In some experiments a plurality of molecules capable of forming the multi-body complex comprise proteins that dimerize with each other, including leucine zippers, alpha helical complexes, etc. In some experiments, a plurality of molecules capable of forming the multi-body complex comprises proteins chemically linked to a small molecule that binds an enzyme. In some experiments the plurality of molecules capable of forming the multi-body complex comprise proteins capable of recruiting other proteins, non-limiting examples of which are proteins that recruit other proteins via peptides/small molecules, such as but not limited to degraders, which are small molecules that bind two different proteins, in which one binds the protein of interest (to be degraded) and the other binds a protein that targets the 3-body complex to the proteosome for degradation (non-limiting examples of such molecules include pthalidomide and synthetic peptides that bind and recruit proteins to the proteome).

In some experiments, the plurality of molecules capable of forming the multi-body complex comprise chemically modified proteins, which in some experiments are proteins chemically modified with small molecules that bind other proteins, non-limiting examples of which are binding via Click chemistry, post-translational modification, non-canonical amino acids, etc.

In some experiments, a first protein of a multi-body complex of interest is a DNA binding domain and is chemically modified such that it was fused to a small molecule that another protein bound tightly to. Studies are performed using DBD modified with biotin (which binds with rpoZ-streptavadin), or the DBD fused to a small molecule drug candidate (such as a kinase inhibitor), and then the rpoZ protein fused to a kinase. In each instance, when the molecules bind, they form a 3-molecule multi-body complex.

### Results

A “sweet spot” of protein binding in the performed experiments is identified, and protein evolution is performed in continuous flow using a combination of small molecules at the sweet spot, or under conditions that favor improved binding (less than the maximum dose effect), or under conditions that favor reduced binding (greater than the maximum dose effect). Results demonstrate effective adjustment of binding is achieved by implementing the conditions that favor improved binding or that favor reduced binding.

### Example 3

Methods of the invention are used to identify equilibrated kinetic inducer conditions, using a liquid handling robot as described in Chory, E. et al., Mol Syst Biol 2021 March; 17(3): e9942. doi: 10.15252/msb.20209942, by equilibrating growth on the liquid handling robot, and then equilibrating expression, following induction with a small molecule.

In some experiments, an evolution circuit is prepared that comprises the following components: (1) the DNA binding protein+antigen (protein fusion) expressed under an inducible promoter (pLac, pBad, etc.), off of a plasmid; (2) the rpoZ protein (RNAP subunit)+antibody (protein fusion) expressed under a different inducible promoter (pLac, pBad, etc.) off of a plasmid or in the genome; (3) RNAP expressed endogenously in bacteria; and (4) various inducer concentrations that control the strength of induction for the various promoters. Conditions that result in an optimized equimolar ratio are determined and when the above components are expressed in the right equimolar ratio, they result in the expression of a protein required for phage proliferation. This study includes either (1) all components that result in the direct expression of the protein pIII, which is required for phage expression; (2) all components that result in the expression of a T7 Polymerase which then drives the expression of pIII which is required for phage expression; (3) all components that result in the expression of one subunit of a split 17 polymerase, which when co-expressed with the other split subunit, drives the expression of pIII, which is required for phage expression. In all cases, pIII can be replaced with another required phage protein (such as pVI, pIV, etc., there are 9 total), or even in another phage. In addition, studies are carried out in which many variations of promoters/polymerases are used in place of the T7RNAP, to control expression of any of the components.

Another study is performed in which an evolution circuit is prepared that comprises, all possible permutations of promoter/antigen/antibody combinations and also the various permutations of inducible RNAP that could also be expressed under inducible control.

Another study is performed in which an evolution circuit is prepared that comprises: (1) the DNA binding protein+surface receptor (protein fusion) expressed under an inducible promoter (pLac, pBad, etc.), off of a plasmid; (2) The rpoZ protein (RNAP subunit)+receptor ligand (protein fusion) expressed under a different inducible promoter (pLac, pBad, etc.) off of a plasmid or in the genome; (3) RNAP expressed endogenously in bacteria; and (4) various inducer concentrations that control the strength of induction for the various promoters. In certain promoters included in the evolution circuit are pLAC (IPTG), pTet (tetracycline inducible), pBAD (arabinose inducible), jEDx (crystal violet inducible), or pPSP (phage shock promoter. Other experiments are performed with an evolution circuit comprising: DBD-ProteinA+rpoZ-ProteinB+RNAP (under all possible promoter plasmids permutations).

### Results

A “sweet spot” of protein binding in the performed experiments is identified, and protein evolution is performed in continuous flow using a combination of small molecules at the sweet spot, or under conditions that favor improved binding (less than the maximum dose effect), or under conditions that favor reduced binding (greater than the maximum dose effect). Results demonstrate effective adjustment of binding is achieved by implementing the conditions that favor improved binding or that favor reduced binding.

### Example 4

Utilization of Model scFv and Dual Promoters in Phage Propagation

Studies were performed in which a model scFv was utilized alongside two distinct inducible promoters. This method involved the application of a dilution series of inducers to regulate the expression levels of the model scFv, facilitating the propagation of phage under a variety of conditions. In one study a pBAD promoter was used as the inducible promoter. In another study a pLac promoter was used as the inducible promoter.

### Materials and Methods

Studies were performed comprising methods described herein including utilizing bacteriophage and PACE to calculate phage propagation rates. In certain experiments, the model scFv was expressed under the control of two inducible promoters (for example a pBAD promoter and a pLac promoter). A range of inducer concentrations was methodically applied in a dilution series to modulate expression levels. This configuration was executed in a high-throughput format, allowing for the analysis of multiple variable conditions concurrently, using methods described herein. These studies utilized new data actually used

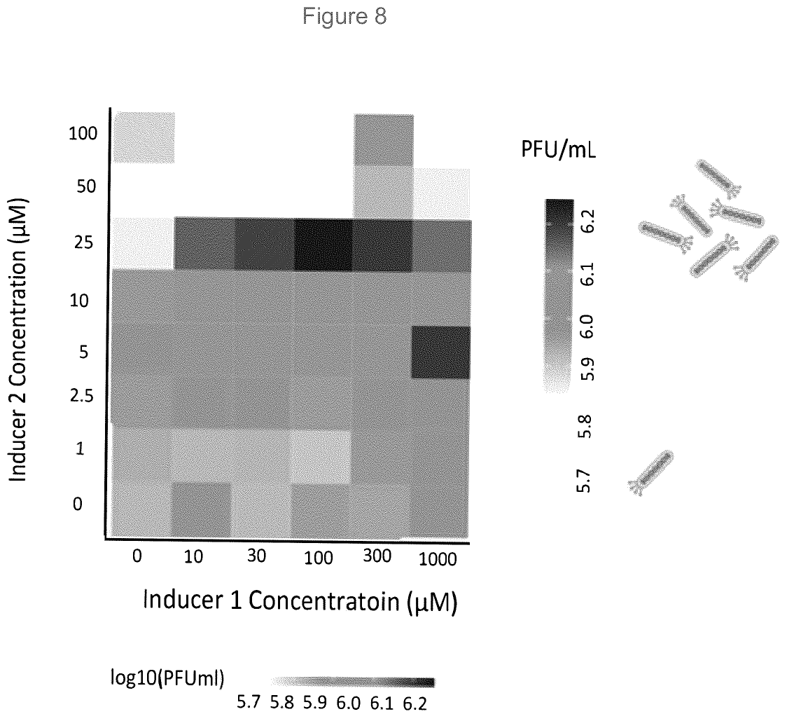

The experiments included phage enrichment in a 6×8 matrix in which two different small molecules were tested across varying concentrations. Each cell in the grid (see FIG. 8) corresponded to a unique combination of concentrations for the two molecules. The data shown are averages of duplicate measurements. Phage enrichment was quantified in terms of plaque-forming units per milliliter (PFU/mL) and was color-coded for ease of interpretation. The color gradient represented the degree of phage enrichment, with darker colors indicating higher levels of enrichment. This visualization highlights the optimal concentration ranges for maximal phage propagation under the given experimental conditions.

### Results

In some instances, a specific inducer concentration was identified at which phage propagation was optimized, denoting an operational “sweet spot.” This concentration provided the most advantageous conditions for phage proliferation, underscoring the efficiency of the dual promoter system in conjunction with the model scFv in high-throughput assays. FIG. 8 shows experimental results obtained in this study.

### Example 5

Selective Amplification of Antibodies with Diverse Affinities in Mixed Phage Populations

Studies were performed using methods for selection of antibodies with varying affinities using a mixed population of phage. This method was used to analyze the competitive dynamics of antibodies with different binding strengths under varied inducer concentrations. Studies were performed comprising methods described herein including utilizing bacteriophage and PACE to calculate phage propagation rates.

### Materials and Methods

In various implementations, a mixture of phage, each harboring antibodies of differing binding strengths, was subjected to a high-throughput assay such as described above herein. The studies comprised certain methods described herein. The high-throughput assay was used to evaluate the competitive interactions among these antibodies under a spectrum of inducer concentrations. In certain studies, methods were used to select anti-GCN4 peptide antibodies with varying affinities. Studies also analyzed competitive dynamics of anti-GCN4 peptide antibodies with different binding strengths under varied inducer concentrations.

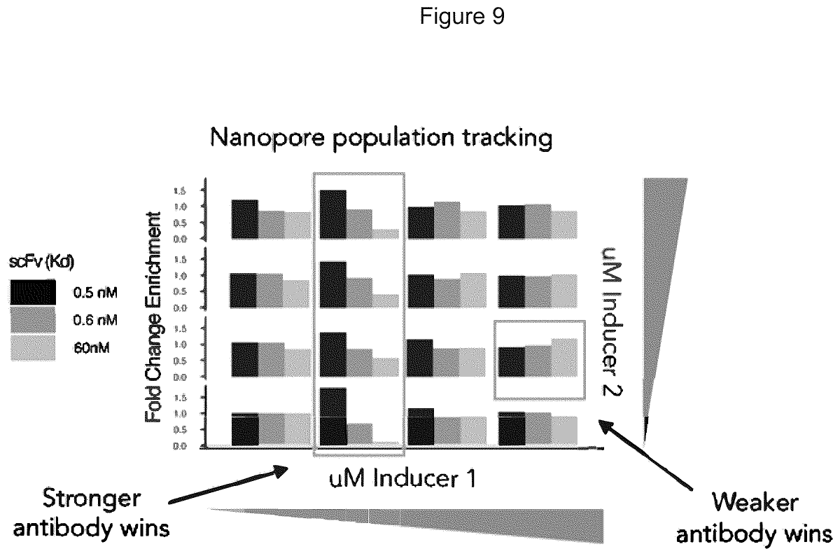

One study included testing three different mixtures of anti-GCN4 peptide antibodies, each having distinct binding affinities (0.5 nM, 0.6 nM, and 60 nM), within a 4×4 concentration matrix. The experiment included sequencing these mixtures using long-read nanopore sequencing to accurately deconvolute the proportion of each antibody type in the population under varying conditions. Results are shown in FIG. 9.

### Results

In certain experiments, at the maximal inducer concentration (identified as the “sweet spot”), the population was dominated by the phage carrying the scFv with the highest binding affinity. Notably, beyond this optimal concentration, a shift in population dynamics was observed, where phage with a weaker scFv became predominant. This observation is pivotal for the selection of antibodies with preferred affinities (rather than just the strongest), highlighting the influence of binding strength in the competitive selection within a heterogenous phage population.

In the study described above, FIG. 9 shows a bar plot illustrating the fraction of the population for the three different mixtures of antibodies, each having distinct binding affinities (0.5 nM, 0.6 nM, and 60 nM). Each bar in FIG. 9 represents the fraction of a specific antibody mixture at a particular concentration combination, providing insights into the competitive dynamics and selection efficiency among antibodies with varying affinities. The use of long-read sequencing allowed for a detailed and precise analysis of the complex interplay within these mixed phage populations.

### EQUIVALENTS

It is to be understood that the methods and compositions that have been described above are merely illustrative applications of the principles of the invention. Numerous modifications may be made by those skilled in the art without departing from the scope of the invention.

Although the invention has been described in detail for the purpose of illustration, it is understood that such detail is solely for that purpose and variations can be made by those skilled in the art without departing from the spirit and scope of the invention, which is defined by the following claims.

The contents of all literature references, publications, patents, and published patent applications cited throughout this application are incorporated herein by reference in their entirety.

What is claimed is:

1. A method of evolving molecules of multi-body complexes, the method comprising:
(a) preparing an evolution circuit comprising a plurality of molecules capable of forming a multi-body complex;
(b) forming the multi-body complex comprising the plurality of molecules in the prepared evolution circuit;
(c) determining for each of the plurality of molecules, one or more equilibrium conditions in the prepared evolution circuit capable of producing an equimolar ratio of each of the plurality of molecules in the evolution circuit;
(d) selecting one or more evolution conditions for the plurality of molecules based at least in part on the one or more equilibrium conditions determined for each of the plurality of molecules;
(e) maintaining the selected one or more evolution conditions; and
(f) evolving one or more of the molecules of the multibody complex under the maintained one or more selected evolution conditions.

2-3. (canceled)

4. The method of claim 1, wherein the plurality of molecules capable of forming the multi-body complex comprises three molecules.

5-6. (canceled)

7. The method of claim 1, wherein preparing the evolution circuit comprises an automated, high-throughput turbidostat method on a liquid-handling robot, optionally wherein the liquid-handling robot comprises a high-through turbidostat comprising a cultured cell for protein evolution, and optionally wherein the cultured cell is bacteria+phage or yeast.

8-10. (canceled)

11. The method of claim 7, further comprising:
equilibrating growth of the cultured cell;
contacting a plurality of portions of the equilibrated cell culture with one or more combinations of concentrations of a first molecule inducing agent and a second molecule-inducing agent;
inducing production of the first molecule of the multi-body complex and the second molecule of the multi-body complex; and
identifying one or more combinations of concentrations of the first molecule-inducing agent and the second molecule-inducing agent that result in kinetic equilibrium of the first molecule and second molecules in the evolution circuit.

12. The method of claim 11, further comprising:
contacting a portion of the equilibrated cell culture with one of the identified combinations of concentrations of the first molecule-inducing agent and the second molecule-inducing agent that results in kinetic equilibrium of the first molecule and second molecules in the evolution circuit with a predetermined concentration of the third molecule of the multi-body complex that results in kinetic equilibrium of the third molecule; and
initiating evolution of one or more of the first, second, and third molecules in the evolution circuit,
optionally wherein the cultured cell is a bacterium, and the method further comprises contacting the cultured cell comprising the first, second, and third molecules of the multi-body complex maintained at kinetic equilibrium with a phage.

13-15. (canceled)

16. The method of claim 7, wherein two of the molecules capable of forming the multi-body complex are inducible proteins, optionally wherein the two inducible proteins are expressed in plasmids in the cultured cell.

17-19. (canceled)

20. The method of claim 7, wherein the one of the molecules capable of forming the multi-body complex is a DNA-directed RNA polymerase (RNAP) expressed by the cultured cell, optionally wherein the RNAP expressed by the cultured cell is endogenous to the cultured cell.

21-22. (canceled)

23. The method of claim 11, wherein the identifying of the one or more conditions resulting in kinetic equilibrium of the first, second, and third molecules comprises:
contacting the cultured cell with a plurality of amounts of the first and second molecule-inducing agents, respectively;
determining an amount of the first molecule inducing agent that induces the kinetic equilibrium of the first molecule; and
determining an amount of the second molecule inducing agent that induces the kinetic equilibrium of the second molecules.

24. The method of claim 11, wherein one or more of the first and second molecule inducing-agents is a small-molecule inducing agent, optionally wherein the small molecule inducing agent is a promoter.

25-26. (canceled)

27. The method of claim 11, wherein one or more of the first and second molecule inducing agents is light.

28. The method of claim 23, wherein the one or more conditions are one or more of: an amount of the inducing agent, identity of the inducing agent, timing of an initial contact of the cultured cell with the inducing agent, length of time of contact of the cultured cell with the inducing agent.

29. The method of claim 1, wherein the produced kinetic equilibrium of the plurality of molecules results in an equimolar ratio of the plurality of molecules capable of forming the multi-body complex.

30. The method of claim 1, wherein the multibody complex further comprises one or more detectable reporter.

31. (canceled)

32. The method of claim 30, wherein the determining of one or more conditions in the prepared evolution circuit capable of producing kinetic equilibrium of each of the plurality of molecules, comprises detecting the detectable reporter.

33. The method of claim 11, wherein one or more of the molecules forming the multi-body complex is a protein and preparing the evolution circuit comprises expressing the one or more proteins in the cultured cell.

34. The method of claim 33, further comprising contacting the cultured cell in the evolution circuit with one or more independently selected inducible promoter to induce expression of the one of more of the proteins of the multi-body complex.

35. The method of claim 34, wherein one of the induced proteins, is endogenously expressed by the cultured cell, optionally wherein the induced protein endogenously expressed by the cultured cell is RNAP.

36. The method of claim 1, wherein the plurality of molecules is four molecules.

37-43. (canceled)

44. A method of evolving one or more molecules of a multi-body complex, the method comprising:
(a) preparing an evolution circuit comprising a plurality of molecules capable of forming a multi-body complex;
(b) determining for each of the plurality of molecules, one or more equilibrium conditions in the prepared evolution circuit capable of producing an equimolar ratio of each of the plurality of molecules in the evolution circuit;
(c) selecting one or more evolution conditions for the plurality of molecules based at least in part on the one or more equilibrium conditions determined for each of the plurality of molecules;
(d) maintaining the selected one or more evolution conditions; and
(e) evolving one or more of the molecules of the multibody complex under the maintained one or more selected evolution conditions.

45. The method of claim 44, further comprising forming the multi-body complex comprising one or more of the evolved molecules.
